(19) United States
(12) Patent Application Publication
(10) Pub. No.: US 2019/0254402 A1
(43) Pub. Date: Aug. 22, 2019

(54) DEVICE COMPRISING AN ABRASIVE SURFACE FOR USE IN PERSONAL CARE

(71) Applicant: THE CURVE AF 2015 APS, Copenhagen Ø (DK)

(72) Inventor: Roberto MACHUCA, Copenhagen K (DK)

(21) Appl. No.: 16/305,188

(22) PCT Filed: Jun. 14, 2017
(86) PCT No.: PCT/EP2017/064624

§ 371 Date: Nov. 28, 2018

(30) Foreign Application Priority Data
Jun. 15, 2016 (DK) PA 2016 00349
Sep. 6, 2016 (DK) PA 2016 00518

(51) Int. Cl. A45D 26/00; A45D 29/14; A61B 17/54

(52) U.S. Cl. CPC A45D 26/0004; A61B 2050/0065; A61B 17/54; A45D 29/14

(57) ABSTRACT

The present invention relates to a device (1) comprising an abrasive material (2) and a connection material (3), wherein the abrasive material (2) is disc shaped and comprise a front-side (4) and a back-side (5), the front-side (4) comprises the abrasive part and the back-side (5) is joined to the connection material (3).

### TECHNICAL FIELD OF THE INVENTION

The present invention relates to a device for removing unwanted skin or hair from the body. In particular the present invention relates to an electrical device comprising a rough surface adapted for depilation or for use in pedicure treatment.

### BACKGROUND OF THE INVENTION

Within the field of depilation and pedicure treatment different devices and methods are used in order to remove hair from the body and to remove dead or hard skin cells, e.g. from the feet. In respect of removing hair from the body the traditionally used methods involve waxing or shaving and many different devices and compositions exists for this purpose, such as shavers, electrical shavers, knives, electrical devices, mechanical devices, lubrications, was applications, lacer technology, chemical applications and abrasive roller devices.

The abrasive roller devices has the disadvantages that the abrasive effect of the rollers gets worn quickly and needs to be changes with a new to provide proper continuing effect. Furthermore, changing the rollers are not quick and easy, which make it cumbersome to the user to continuously changing between the rollers so that the roughness of the roller fits to the hardness, and/or thickness of dead or hard skin cells to be removed and considering the sensitivity of the part to be treated.

Furthermore, traditionally the abrasive roller devices are to be used on dry skin and have a rotating speed above 2000 rpm (rounds per minute). Traditional abrasive roller devices need to be used on dry skin and/or use a rotation speed above 2000 rpm in order to provide proper effect. However, this also has a consequence on the roller, which is quickly worn and when used the costumer must be very careful as, in particular the high speed may give burns, and painful areas on the treated parts.

Hence, an improved device for remove hair from the body and to remove dead or hard skin cells, e.g. from the feet would be advantageous, and in particular a more efficient and/or reliable device which is easy to use, easy to switch between different roughness, avoiding or reducing the risk of burns, discomfort and skin irritation, and providing a silky smooth surface after the treatment would be advantageous.

### SUMMARY OF THE INVENTION

Thus, an object of the present invention relates to device for removing hair from the body and/or removing dead or hard skin cells, e.g. from the feet.

In particular, it is an object of the present invention to provide a device for removing hair from the body and/or removing dead or hard skin cells, e.g. from the feet that solves the above-mentioned problems of the prior art with wear, handling of the device when switching between the abrasive parts, burns and skin irritation.

Thus, one aspect of the invention relates to a device (1) comprising an abrasive material (2) and a connection material (3), wherein the abrasive material (2) is disc shaped and comprise a front-side (4) and a back-side (5), the front-side (4) comprises the abrasive part and the back-side (5) is joined to the connection material (3).

Another aspect of the present invention relates to the use of a device according to the present invention for a pedicure treatment.

Yet another aspect of the present invention relates to the use of a device according to the present invention for removing hair from the body.

### BRIEF DESCRIPTION OF THE FIGURES

FIG. 1 shows a device (1) comprising an abrasive material (2) and a connecting material (3) according to the present invention, to be connected with a housing (not shown) and e.g. used for pedicure treatment or as an epilator.

FIG. 2 shows a device (1) according to the present invention, comprising an abrasive material (2) and a connecting material (3). The connecting material (3) is connected with a housing (8). The device (1) shown in FIG. 2 may be used as an epilator, removing hair from the body.

FIG. 3 shows a device (1) according to the present invention, comprising an abrasive material (2) and a connecting material (3). The connecting material (3) is connected with a housing (8). The device (1) shown in FIG. 2 may be used as in pedicure treatment, e.g. removing dead skin cells, preferably, from the feet.

The present invention will now be described in more detail in the following.

### DETAILED DESCRIPTION OF THE INVENTION

The devices know today may be either painful, introduce wounds, have a very bad durability, difficult to use, for professional application or may provide insufficient results. The inventor of the present invention surprisingly found that the device according to the present invention meets the mentioned problems with the prior art and provided a device for remove hair from the body and/or to remove dead or hard skin cells, e.g. from the feet with is more efficient, easy to use, easy to switch between different roughness, without burns, discomfort and skin irritation, and resulting in a silky-smooth surface after the treatment.

An aspect of the present invention relates to a device (1) comprising an abrasive material (2) and a connection material (3), wherein the abrasive material (2) is disc shaped and comprise a front-side (4) and a back-side (5), the front-side (4) comprises the abrasive part and the back-side (5) is joined to the connection material (3).

In the context of the present invention, the term “abrasive material” relates to a material that may be used to remove hair from the body and/or to remove dead or hard skin cells, e.g. from the feet by friction, e.g. through the action of rubbing or peeling or scratching. The treated surface may, after treatment using the abrasive material, become silky smooth.

In a preferred embodiment of the present invention the abrasive material may be made of a silicate material, a ceramic material, a plastic material or a combination hereof. Preferably, the abrasive material may be made of a silicate material.

In a further embodiment of the present invention the silicate material may be glass, flat glass, pressed glass or hardened glass.

In order to improve the stability of the abrasive material and to improve the user safety in case og breakage or if accidentally dropped, the silicate material may preferably be made of or have a glass body formed from hardened glass.

The abrasive material comprises a front side where the abrasive part is places. This abrasive part is responsible for the effect of removing hair from the body and/or for removing dead or hard skin cells, e.g. from the feet, by friction, e.g. through the action of rubbing or peeling or scratching.

In an embodiment of the present invention the abrasive part has a roughness in the range of 5-150 μm, such as in the range of 10-100 μm, e.g. in the range of 20-90 μm, such as in the range of 30-80 μm, e.g. in the range of 40-70 μm, such as in the range of 50-60 μm, e.g. in the range of 60-90 μm, such as in the range of 10-30 μm.

The roughness of the abrasive part may be selected according to the intended use, skin sensitivity, material to be removed (hair or skin or the hardness of the skin).

In an embodiment of the present invention the abrasive part may be provided by chemical treatment, such as etching, and/or mechanical treatment, such as sanding.

In an alternative embodiment of the present invention the abrasive part comprises a smooth surface (e.g. comprising a silicate material, a ceramic material, or a plastic material) having particles attached. Preferably, the particles may be selected from the group consisting of sand, glass, plastic or metal.

In an embodiment of the present invention the device further comprises a housing (8).

The connecting material may be provided to ensure the connection between the abrasive material and a housing.

In an embodiment of the present invention the abrasive material (2) and the connection part (3) may be made of the same materials, made of different materials, or made in one material.

In the present invention, the term “made in one material” relates to a single piece comprising the abrasive material and the connecting material.

In an embodiment of the present invention the abrasive material (2) and the connecting part (3) are made in one material by moulding, casting, or 3-D printing.

When the abrasive material and the connecting material have not been not made in one material they comprise a combination of separate element which are joined together.

In an embodiment of the present invention the back-side (5) of the abrasive material (2) and the attaching part (6) of the connecting part (3) are joined, preferably by gluing.

In another embodiment of the present invention the abrasive material (2) may comprise two abrasive parts, preferably an abrasive front-side (4) and an abrasive back-side (5). The advantage of having both an abrasive front-side (4) and an abrasive back-side (5) is that the abrasive material (2) may comprise two different roughness.

Preferably the abrasive material (2) comprising the two abrasive parts, i.e. an abrasive front-side (4) and an abrasive back-side (5), may be turned to change the desired roughness or if both sides of the abrasive material (3) has the same roughness, and if/when one side of the abrasive material (2) has been worn.

In an embodiment of the present invention, the attaching part (6) of the connecting part (3) comprises gripping elements to fix the abrasive material (2) to the attaching part (6). Preferably, the attaching part (6) of the connecting part (3) comprises one or more gripping elements to fix the abrasive material (2), e.g. 3 or more gripping elements, such as 4 or more gripping elements, e.g. 5 or more gripping elements, such as 6 or more gripping elements.

Preferably, the gripping elements may not extent further forward than the abrasive front-side (4) or the abrasive back-side (5)

In one embodiment of the present invention, the edge of the abrasive material (2) may comprise a curve structure, allowing engagement with the gripping elements.

Preferably, the length of the gripping elements is shorter than the thickness of the abrasive element.

In the event of one gripping element the of the attaching part (6), the gripping element may be a ring to be irreversible attached to the attaching part (6) and to be reversible attached to the abrasive material (2) allowing the abrasive material (2) to be released and turned.

In an embodiment of the present invention the gripping elements comprises pads (e.g. silicone or plastic), suitable to fix the abrasive material (2) to the attaching part (6).

In a further embodiment of the present invention the connecting material comprises an attaching part (6) and a coupling part (7).

In an even further embodiment of the present invention, the housing (8) may be connected to the abrasive material (2) via the coupling part (7) of the connecting material (3).

In an even further embodiment of the present invention the coupling part (7) may be provided with a tongue (10) to engage with a groove (11) on the housing.

Preferably, the groove (11) may be provided with holding means to ensure the tongue leaves the groove simply by gravity. In an embodiment of the present invention, the holding means are a ball, a spring, a hinge or a magnet.

The housing according to the present invention may have the function as a handle and/or an electrical device providing the moving action to the abrasive material.

In an embodiment of the present invention wherein the housing (8) comprises electrical means (9) for electrically moving the abrasive material (2). Preferably, the electrical means (9) for electrically moving the abrasive material (2) provides a axial rotation around the centreline through the front-side (4) and the back-side (5), of the abrasive material.

In another embodiment of the present invention the housing (8) comprises a battery connected to the electrical means (9).

The housing may preferably be made or a water-resistant material and all connecting areas may be sealed. Hence the device and/or the housing according to the present invention may preferably, be waterproof.

In the present invention, the term “waterproof” relates to a device and/or a housing which is Impervious to or unaffected by water. This waterproof effect may be obtained by make the housing or use a coating or treat the housing with or prepare the housing from rubber, plastic, or a sealing agent to prevent penetration by water.

In order to improve performance during operation of the device according to the present invention, the angle between the direction of the housing (8) and the front side (4) of the abrasive material (2) may be fixed. In an embodiment of the present invention the angle between the direction of the housing (8) and the front side (4) of the abrasive material (2) is in the range of 0-90°, such as in the range of 5-50°, e.g. in the range of 7.5-30°, such as in the range of 10-25°, e.g. in the range of 12-20°, such as in the range of 10-15°.

In the context of the present invention the term “direction of the housing” relates to the longitudinal direction of the housing, preferably, with the abrasive material in one end of the device.

In another embodiment of the present invention the angle between the direction of the housing (8) and the front side (4) of the abrasive material (2) may be adjustable allowing the user to provide the best contact and position during operation. Hence, the front side (4) of the abrasive material (2) may be movable relative to the direction of the housing (8) in an angle between 0-90°, such as an angle between 5-50°, e.g. an angle between 7.5-30°, such as an angle between 10-25°, an angle between 12-20°, such as an angle between 0-15°.

In the context of the present invention, the term “movable relative to” relates to the angle between the front side (4) of the abrasive material (2) and the direction of the housing (8) may be adjustable.

In order to reduce or avoid the risk of burns, skin irritation, wear and other damages the speed of the abrasive material, the rounds per minute (rpm) should be kept low.

In an embodiment of the present invention the electrical means (9) may be configured to generate a speed of the abrasive material (2) in the range of 100-2000 rpm, such as in the range of 200-1750 RPM, e.g. in the range of 300-1500, such as in the range of 400-1250 RPM, e.g. in the range of 500-1000, such as in the range of 550-750 RPM, e.g. in the range of 600-700.

The device according to the present invention may be a personal care device.

In an embodiment of the present invention the personal care device is an epilator. Preferably, the epilator has electrical means (9) configured to generate a speed of the abrasive material (2) in the range of 100-2000 rpm, such as in the range of 200-1750 RPM, e.g. in the range of 300-1500, such as in the range of 400-1250 RPM, e.g. in the range of 500-1000, such as in the range of 550-750 RPM, e.g. in the range of 600-700.

In a further embodiment of the present invention the personal care device is an epilator. Preferably, the epilator has electrical means (9) configured to generate a speed of the abrasive material (2) in the range of 50-1500 rpm, such as in the range of 100-1000 RPM, e.g. in the range of 200-750, such as in the range of 300-700 RPM, e.g. in the range of 400-600, such as in the range of 450-550 RPM.

In another embodiment of the present invention personal care device is for pedicure treatment. Preferably, the pedicure treatment has electrical means (9) configured to generate a speed of the abrasive material (2) in the range of 100-2000 rpm, such as in the range of 200-1750 RPM, e.g. in the range of 300-1500, such as in the range of 400-1250 RPM, e.g. in the range of 500-1000, such as in the range of 550-750 RPM, e.g. in the range of 600-700.

In yet another embodiment of the present invention personal care device is for pedicure treatment. Preferably, the pedicure treatment has electrical means (9) configured to generate a speed of the abrasive material (2) in the range of 100-2000 rpm, such as in the range of 300-1750 RPM, e.g. in the range of 500-1500, such as in the range of 750-1250 RPM, e.g. in the range of 800-1200, such as in the range of 900-1100 RPM, e.g. in the range of 950-1050.

In order to provide a proper abrasive effect, the device according to the present invention and/or the electrical means (9) may be configured to generate a torque in the range of 1.900-2.200 kg/cm, such as in the range of 2.000-2.100 kg/cm, e.g. in the range of 2.010-2.050, such as in the range of 2.011-2.030 kg/cm, e.g. in the range of 2.012-2.020, such as about 2.014.

In an embodiment of the present invention the abrasive material (2) may have a diameter in the range of 550-750 mm and a thickness in the range of 5.5-6.5 mm, a roughness in the range of 5-150 μm, an angle between the direction of the housing (8) and the front side (4) of the abrasive material (2) of 0-15° and wherein the electrical means (9) is configured to generate a torque in the range of 1.900-2.200 kg/cm, such as in the range of 2.000-2.100 kg/cm, e.g. in the range of 2.010-2.050, such as in the range of 2.011-2.030 kg/cm, e.g. in the range of 2.012-2.020, such as about 2.014.

I yet an embodiment of the present invention, the electrical means (9) may be configured to generate a torque in the range of 1.900-2.200 kg/cm, such as in the range of 2.000-2.100 kg/cm, e.g. in the range of 2.010-2.050, such as in the range of 2.011-2.030 kg/cm, e.g. in the range of 2.012-2.020, such as about 2.014, and the electrical means (9) may be configured to generate a speed of the abrasive material (2) in the range of 100-2000 rpm, such as in the range of 200-1750 RPM, e.g. in the range of 300-1500, such as in the range of 400-1250 RPM, e.g. in the range of 500-1000, such as in the range of 550-750 RPM, e.g. in the range of 600-700.

In a preferred embodiment, the device according to the present invention, may be used for a pedicure treatment.

In the context of the present invention, the term “pedicure treatment” relates to a therapeutic treatment, in particular for the feet. The treatment may be used to remove dead skin, soften and remove hard skin and shapes and treating toenails.

In an embodiment of the present invention the angle between the direction of the housing (8) and the front side (4) of the abrasive material (2), of a device to be used for pedicure treatment may be fixed or may be adjustable. Preferably, the front side (4) of the abrasive material (2) relative to the direction of the housing (8) of a device to be used for pedicure treatment may be in an angle between 0-90°, such as an angle between 5-50°, e.g. an angle between 7.5-25°, such as an angle between 8-20°, an angle between 10-15°, such as an angle between 0-10°, e.g. about 0.

In a preferred embodiment, the device according to the present invention may be used as an epilator for removing hair from the body.

In a further embodiment of the present invention, the angle between the direction of the housing (8) and the front side (4) of the abrasive material (2), of a device to be used as an epilator, may be fixed or may be adjustable. Preferably, the front side (4) of the abrasive material (2) relative to the direction of the housing (8) of a device to be used for pedicure treatment may be in an angle between 0-90°, such as an angle between 5-50°, e.g. an angle between 7.5-30°, such as an angle between 10-25°, an angle between 12-20°, such as about 15°.

Preferably the abrasive material (2) has a diameter in the range of 10-100 mm, such as in the range of 20-90 mm, e.g. in the range of 30-80 mm, such as in the range of 40-70 mm, e.g. in the range of 50-60 mm.

Preferably the abrasive material (2) has a thickness in the range of 1-11 mm, such as in the range of 2-10 mm, e.g. in the range of 3-9 mm, such as in the range of 4-8 mm, e.g. in the range of 5.5-65 mm.

It should be noted that embodiments and features described in the context of one of the aspects of the present invention also apply to the other aspects of the invention.

The invention will now be described in further details in the following non-limiting descriptions of the figures.

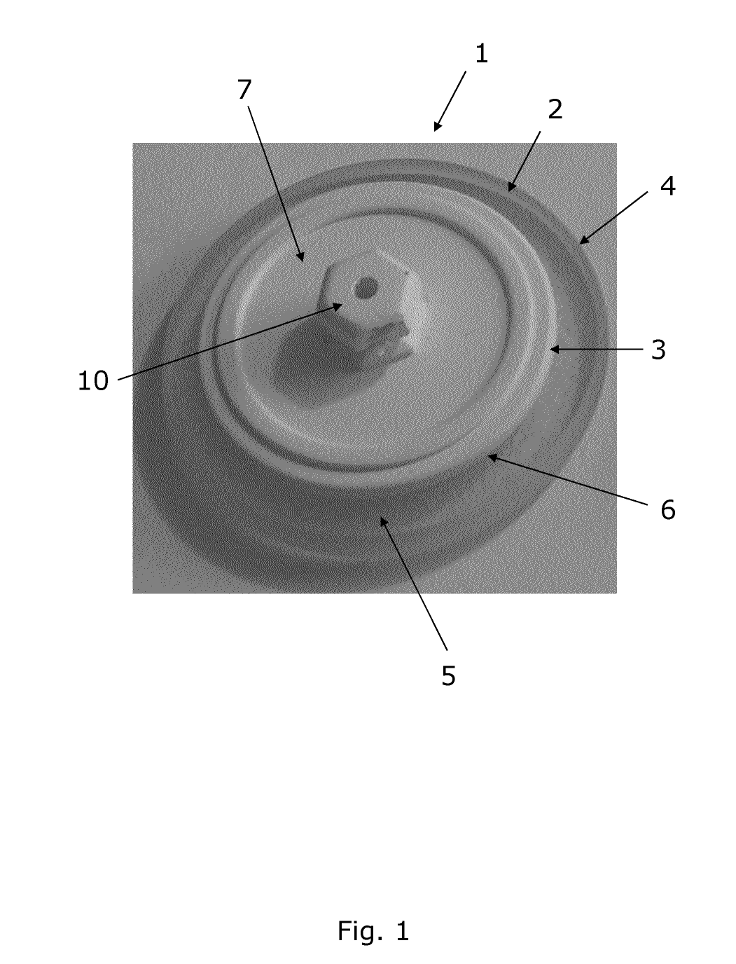

FIG. 1 illustrates a device (1) according to the present invention. The device comprises a disc shaped abrasive material (2) having a front-side (4) and a back-side (5). The abrasive material (2) being joined with a connection material (3) via the back-side (5) of the abrasive material (2), which preferably is a smooth surface, and connected to the attaching part (6) of the connection material (3). The abrasive material (2) may be made of a silicate material, a ceramic material, a plastic material or a combination hereof. Preferably, the abrasive material (2) is made of a silicate material, such as glass, flat glass, pressed glass or hardened glass. The abrasive material (2) may be joined with the connection material (3) in many different ways, preferably by gluing. The connection material (3) is preferably provided with a tongue (10) located on the coupling part (7) of the connection material (3). The tongue (10) may be introduced into a groove (not shown) of a housing (not shown) to provide an electrical movement, rotating movement of the abrasive material (2).

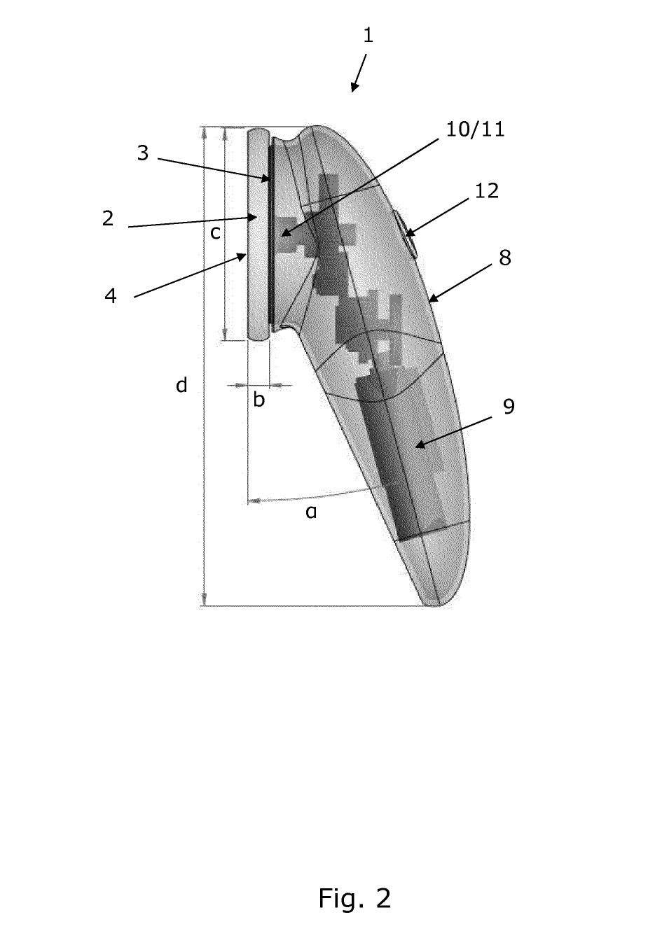

FIG. 2 shows a side view of the device (1) according to the present invention. The device (1) comprises an abrasive material (2) and a connecting material (3). The connection material (3) is connected, to a housing (8) via a tongue (10) which engage with a groove (11) and which transmits the movement from the housing to a rotating movement of the abrasive material (2). The movement in the housing (8) and of the abrasive material (2) may be provided by electrical means (9), such as a battery. In order to provide the best handling position when in use, the angle between the direction of the housing (8) and the front side (4) of the abrasive material (2), may be fixed or may be adjustable to an angle between 0-90° preferably the angle α is about 15°. The dimensions of the abrasive material (2) may be selected to optimise durability and strength, but at the same time considering weight of the device (1) and handling during use. The device (1) may be turned on or turned off by the activation button (12). In one embodiment the diameter (c) of the abrasive material (2) is 60 mm, the width of the abrasive material (2) is about 6 mm, and the length of the device (1) is about 135 mm. The ratio between diameter of the abrasive material (2) and the thickness of the abrasive material (2) is preferably in the range of 2:1 to 20:1, such as in the range of 5:1 to 15:1, e.g. in the range of 7:1 to 12:1, such as a ratio about 10:1. The device (1) shown in FIG. 2 may preferably be used as an epilator, removing hair from the body.

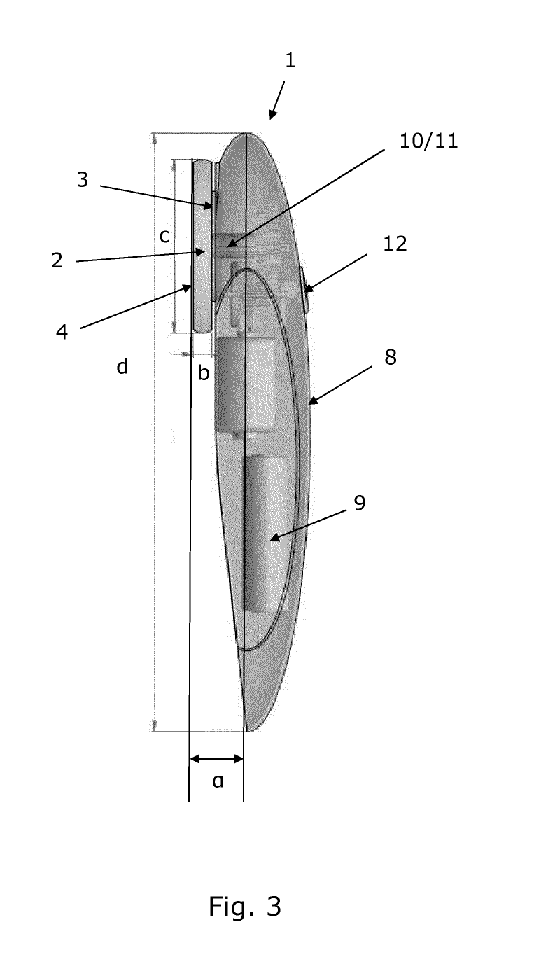

FIG. 3 shows a side view of the device (1) according to the present invention. The device (1) comprises an abrasive material (2) and a connecting material (3). The connection material (3) is connected, to a housing (8) via a tongue (10) which engage with a groove (11) and which transmits the movement from the housing to a rotating movement of the abrasive material (2). The movement in the housing (8) and of the abrasive material (2) may be provided by electrical means (9), such as a battery. In order to provide the best handling position when in use, the angle between the direction of the housing (8) and the front side (4) of the abrasive material (2), may be fixed or may be adjustable to an angle between 0-90° preferably the angle α is about 0°. The dimensions of the abrasive material (2) may be selected to optimise durability and strength, but at the same time considering weight of the device (1) and handling during use. The device (1) may be turned on or turned off by the activation button (12). In one embodiment, the diameter (c) of the abrasive material (2) is 55 mm, the width of the abrasive material (2) is about 6 mm, and the length of the device (1) is about 190 mm. The ratio between diameter of the abrasive material (2) and the thickness of the abrasive material (2) is preferably in the range of 2:1 to 20:1, such as in the range of 5:1 to 15:1, e.g. in the range of 7:1 to 12:1, such as a ratio about 10:1. The device (1) shown in FIG. 2 may be used as in pedicure treatment, e.g. removing dead skin cells, preferably, from the feet.

1-26. (canceled)

27. A device comprising an abrasive material and a connection material, wherein the abrasive material is disc shaped and comprise a front-side and a back-side, the front-side comprises the abrasive part and the back-side is joined to the connection material.

28. The device according the claim 27, wherein the connecting material comprises an attaching part and a coupling part.

29. The device according to claim 27, wherein the abrasive material is made of a silicate material, a ceramic material, a plastic material or a combination hereof.

30. The device according to claim 29, wherein the silicate material is glass, flat glass, pressed glass or hardened glass.

31. The device according to claim 27, wherein the abrasive part has a roughness in the range of 5-150 μm.

32. The device according to claim 27, wherein the abrasive material and the connecting part are made in one material by molding, casting, or 3-D printing.

33. The device according to claim 27, wherein a back-side of the abrasive material and the attaching part of the connecting part are joined.

34. The device according to claim 27, wherein the abrasive part is provided by chemical treatment and/or mechanical treatment.

35. The device according to claim 27, wherein the abrasive part comprises a smooth surface having particles attached.

36. The device according to claim 35, wherein particles are selected from the group consisting of sand, glass, plastic or metal.

37. The device according to claim 27, wherein the device further comprises a housing.

38. The device according to claim 37, wherein the housing is connected to the abrasive material via the coupling part of the connecting material.

39. The device according to claim 37, wherein housing comprises electrical means for electrically moving the abrasive material.

40. The device according to claim 39, wherein the housing comprises a battery connected to the electrical means.

41. The device according to claim 37, wherein the housing is waterproof.

42. The device according to claim 37, wherein an angle between the direction of the housing and the front side of the abrasive material is in the range of 5-90°.

43. The device according to claim 37, wherein the front side of the abrasive material is movable relative to the direction of the housing in an angle between 5-90°.

44. The device according to claim 39, wherein the electrical means is configured to generate a speed of the abrasive material in the range of 100-2000 rpm.

45. The device according to claim 27, wherein the device is a personal care device.

46. The device according to claim 45, wherein the personal care device is an epilator.
